(19) United States
(12) Patent Application Publication
(10) Pub. No.: US 2024/0285444 A1
(43) Pub. Date: Aug. 29, 2024

(54) DIAPER SENSOR, DISPOSABLE DIAPER, AND DISPOSABLE DIAPER KIT

(71) Applicants: NIPPON MEKTRON, LTD., Tokyo (JP); NOK CORPORATION, Tokyo (JP)

(72) Inventors: Azumi TOMINAGA, Tokyo (JP); Kyosuke NAKADA, Fujisawa-shi (JP)

(73) Assignees: NIPPON MEKTRON, LTD., Tokyo (JP); NOK CORPORATION, Tokyo (JP)

(21) Appl. No.: 18/563,658

(22) PCT Filed: Nov. 29, 2022
(86) PCT No.: PCT/JP2022/043883

§ 371 Date: Nov. 22, 2023

(30) Foreign Application Priority Data
Feb. 24, 2022 (JP) 2022-026929

(51) Int. Cl. A61F 13/42

(52) U.S. Cl. CPC A61F 13/42; A61F 2013/424

(57) ABSTRACT

Provided is a paper diaper having a structure capable of more reliably sensing a state of a diaper sensor being soaked in liquid. A diaper sensor 100 includes a sheet-shaped wetness detection sensor device 90 having a circuit 60 breakable or damageable when getting wet with liquid. The diaper sensor 100 includes a highly water-permeable sheet 85 forming the surface of the diaper sensor 100 arranged on a user's body surface side, and at least any one of a first water-absorbing sheet 81 interposed between the highly water-permeable sheet 85 and the wetness detection sensor device 90 or a second water-absorbing sheet 83 arranged on the opposite side of the wetness detection sensor device 90 from the highly water-permeable sheet 85 side.

### TECHNICAL FIELD

The present invention relates to a diaper sensor, a paper diaper, and a paper diaper kit.

### BACKGROUND ART

Patent Literature 1 describes a paper diaper body and a diaper sensor (described as a liquid detection sensor in this literature) provided at a portion of the diaper body corresponding to an external urethral opening.

### CITATION LIST

### Patent Literature

PATENT LITERATURE 1: JP-A-2021-38971

### SUMMARY OF INVENTION

### Problems to be Solved by Invention

According to study conducted by the inventors of the present application, the diaper sensor of Patent Literature 1 has room for improvement in reliability in sensing a state of the diaper sensor being soaked in liquid.

The present invention has been made in view of the above-described problems, and provides a paper diaper having a structure capable of more reliably sensing a state of a diaper sensor being soaked in liquid.

### Solution to Problems

According to the present invention, provided is a diaper sensor including a sheet-shaped wetness detection sensor device having a circuit breakable or damageable when getting wet with liquid. The diaper sensor includes: a highly water-permeable sheet forming a surface of the diaper sensor arranged on a user's body surface side; and at least any one of a first water-absorbing sheet interposed between the highly water-permeable sheet and the wetness detection sensor device or a second water-absorbing sheet arranged on an opposite side of the wetness detection sensor device from a highly water-permeable sheet side.

Further, according to the present invention, provided is a paper diaper including: a paper diaper body: a first diaper sensor provided at a portion of the paper diaper body corresponding to an external urethral opening; and a second diaper sensor provided at a portion of the paper diaper body different from that for the first diaper sensor, in which each of the first diaper sensor and the second diaper sensor is the diaper sensor according to the present invention.

Moreover, according to the present invention, provided is a paper diaper kit including: the paper diaper according to the present invention; and an incontinence pad, the incontinence pad has an incontinence pad body and a pad-side sensor device provided on the incontinence pad body, and the pad-side sensor device is the diaper sensor according to the present invention.

### Effects of Invention

According to the present invention, the state of the diaper sensor being soaked in liquid can be more reliably sensed.

### BRIEF DESCRIPTION OF DRAWINGS

FIG. 1 is an end view of a paper diaper in an embodiment, FIG. 1 showing a diaper sensor and a peripheral structure thereof.

FIG. 2 is an exploded end view of the paper diaper in the embodiment, FIG. 2 showing the diaper sensor and the peripheral structure thereof.

FIG. 3 is a plan view of the diaper sensor in the embodiment.

FIG. 4 is a plan view of a board of the diaper sensor in the embodiment.

FIG. 5 is a plan view of a soluble layer of the diaper sensor in the embodiment.

FIG. 6 is a schematic development view of the paper diaper in the embodiment.

FIG. 7 is an end view of a paper diaper in a modification of the embodiment, FIG. 7 showing a diaper sensor and a peripheral structure thereof.

FIG. 8 is an exploded end view of the paper diaper in the modification of the embodiment, FIG. 8 showing the diaper sensor and the peripheral structure thereof.

### DESCRIPTION OF EMBODIMENTS

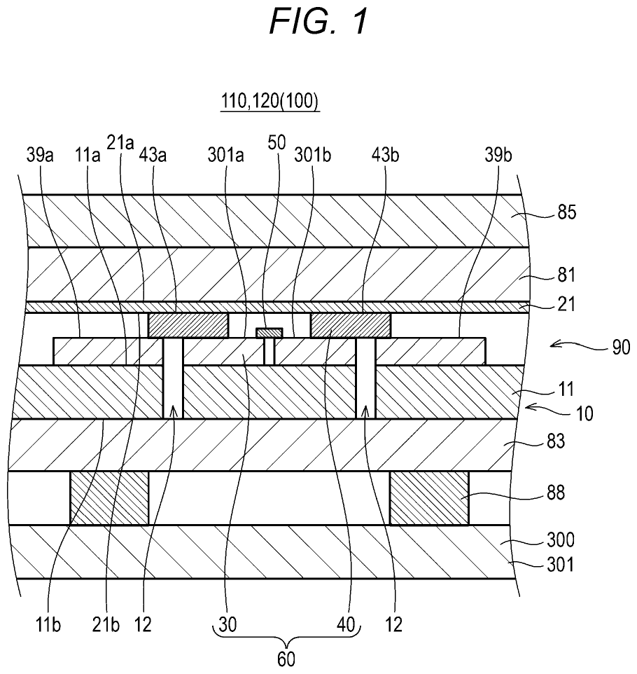

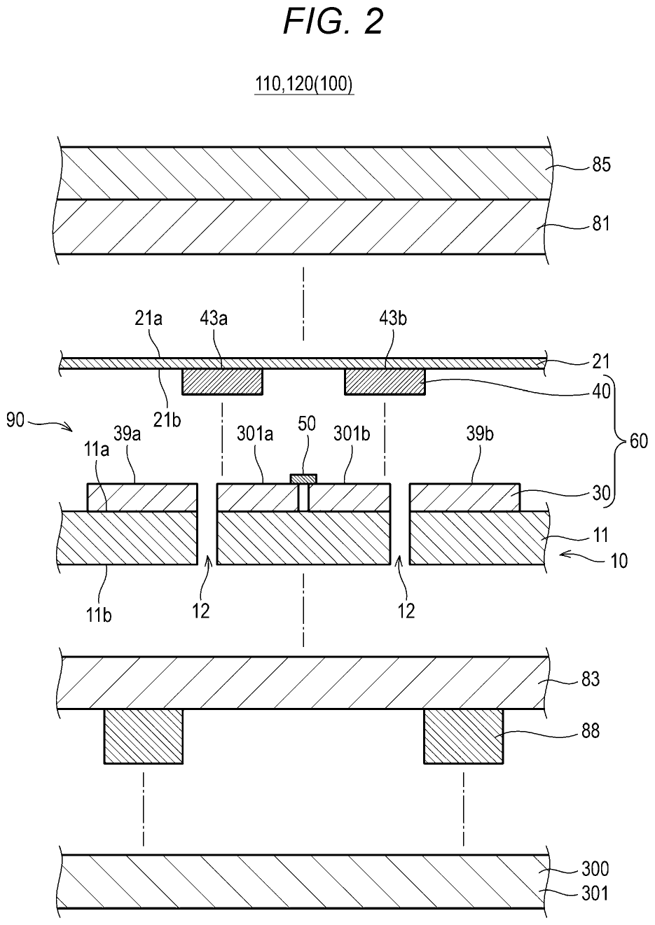

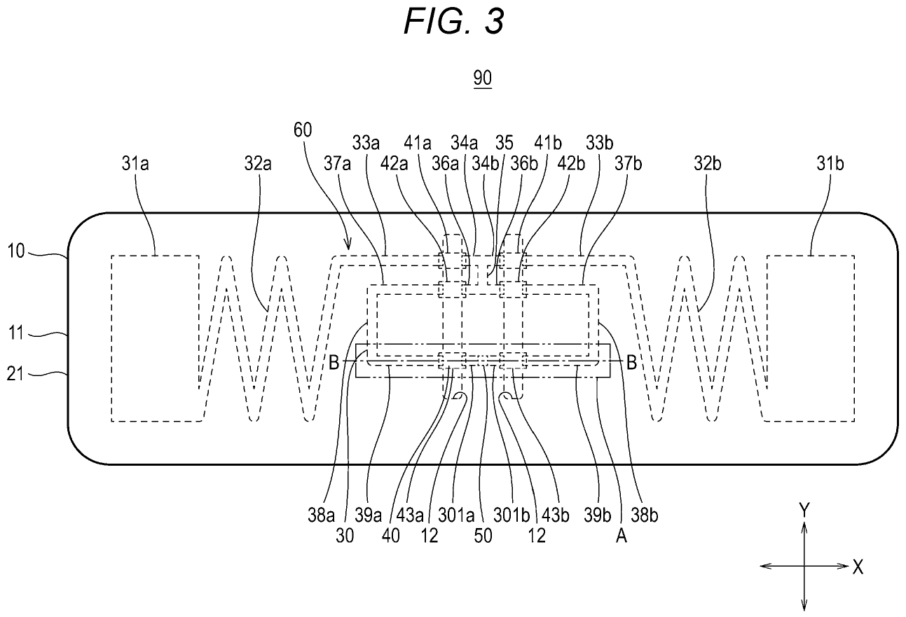

Hereinafter, an embodiment of the present invention will be described using FIGS. 1 to 6. Note that in all the drawings, the same reference numerals are used to represent similar components and description thereof will be omitted as necessary. FIG. 1 shows a cut end surface along a B-B line in a region A (region surrounded by a chain double-dashed frame) shown in FIG. 3, and FIG. 2 is an exploded end view of a portion corresponding to that shown in FIG. 1.

As shown in FIGS. 1 and 2, a diaper sensor 100 (in the case of the present embodiment, first diaper sensor 110 and second diaper sensor 120 as described later) according to the present embodiment includes a sheet-shaped wetness detection sensor device 90 having a circuit 60 breakable or damageable when getting wet with liquid. The diaper sensor 100 includes a highly water-permeable sheet 85 forming a surface of the diaper sensor 100 arranged on a user's body surface side and at least any one of a first water-absorbing sheet 81 interposed between the highly water-permeable sheet 85 and the wetness detection sensor device 90 or a second water-absorbing sheet 83 arranged on the opposite side of the wetness detection sensor device 90 from the highly water-permeable sheet 85 side.

Each of the first water-absorbing sheet 81 and the second water-absorbing sheet 83 is higher in water absorption properties (has better water absorption properties) than the highly water-permeable sheet 85.

According to the present embodiment, since the diaper sensor 100 includes at least any one of the first water-absorbing sheet 81 or the second water-absorbing sheet 83, the first water-absorbing sheet 81 or the second water-absorbing sheet 83 can quickly absorb liquid (urine) and supply the liquid to the wetness detection sensor device 90. Thus, the circuit 60 can be more reliably broken or damaged when the diaper sensor 100 is soaked in liquid. That is, a state of the diaper sensor 100 being soaked in liquid can be more reliably sensed.

In addition, since the diaper sensor 100 includes the highly water-permeable sheet 85 forming the surface of the diaper sensor 100 arranged on the user's body surface side, the highly water-permeable sheet 85 allows, when a user urinates, the urine of the user to quickly penetrate the highly water-permeable sheet 85 toward the circuit 60. With this configuration, the state of the diaper sensor 100 being soaked in liquid can also be more reliably sensed. In addition, since the highly water-permeable sheet 85 forms the surface of the diaper sensor 100 arranged on the user's body surface side, a feeling of smoothness of the surface of the diaper sensor 100 on the user's body surface side can be maintained and a user's uncomfortable feeling can be reduced in the structure including the highly water-permeable sheet 85.

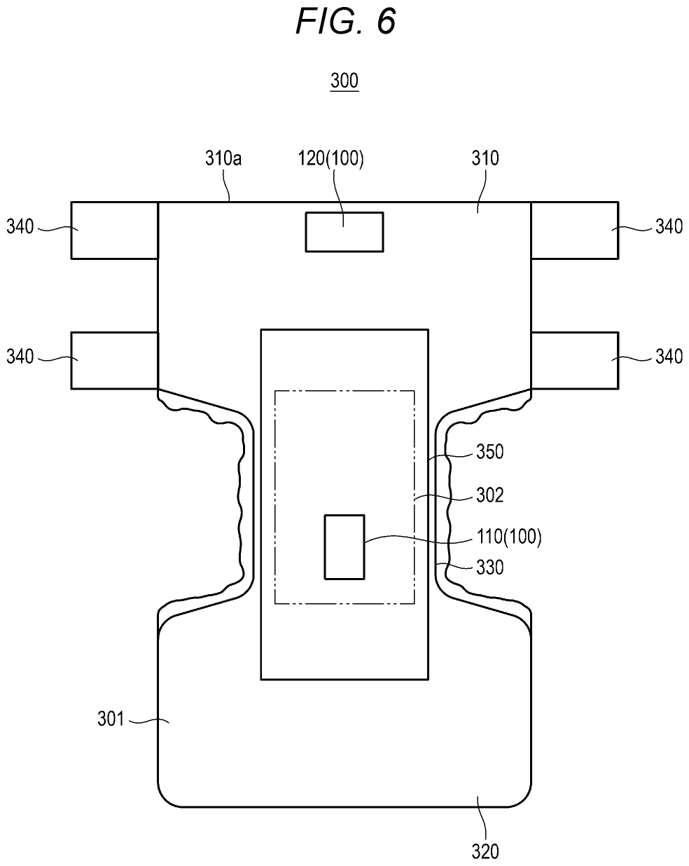

As shown in FIGS. 1 to 6, a paper diaper 300 according to the present embodiment includes a paper diaper body 301 (see FIG. 6), the first diaper sensor 110 provided at a portion 302 (see FIG. 6) of the paper diaper body 301 corresponding to an external urethral opening, and the second diaper sensor 120 provided at a portion of the paper diaper body 301 different from that for the first diaper sensor 110.

Each of the first diaper sensor 110 and the second diaper sensor 120 is the above-described diaper sensor.

Note that in FIG. 3, the first water-absorbing sheet 81, the second water-absorbing sheet 83, and the highly water-permeable sheet 85 are not shown. Moreover, in FIG. 6, the portion 302 of the paper diaper body 301 corresponding to the external urethral opening is indicated by a chain double-dashed line.

According to the present embodiment, the paper diaper 300 includes the first diaper sensor 110 provided at the portion of the paper diaper body 301 corresponding to the external urethral opening and the second diaper sensor 120 provided at the portion of the paper diaper body 301 different from that for the first diaper sensor 110.

With this configuration, when the user urinates in the paper diaper 300, urination of the user can be detected by the first diaper sensor 110. Further, the presence of the urine at a portion other than a portion of the paper diaper 300 corresponding to the external urethral opening can be detected by the second diaper sensor 120. Thus, for example, in a case where the urine of the user leaks out of the paper diaper body 301 or is about to leak out of the paper diaper body 301 (hereinafter merely referred to as “urine leakage”), such urine leakage can be detected by the second diaper sensor 120.

That is, according to the present embodiment, urination of the user and the urine leakage from the paper diaper body 301 can be separately detected.

Hereinafter, in description of, e.g., a positional relationship among the components of the paper diaper 300, the upper side in FIG. 1 will be referred to as an upper side or above, and the opposite side thereof will be referred to as a lower side or below, for example. Moreover, a direction perpendicular to the up-down direction will be described as a horizontal direction, for example. However, these directions are defined for the sake of convenience, and are not intended to limit directions upon manufacturing or use of the paper diaper 300.

The right-left direction in FIGS. 1 to 3 will be referred to as an X-direction, and the up-down direction in FIG. 3 will be referred to as a Y-direction. The X-direction and the Y-direction are directions (horizontal directions) parallel with the planar direction of the paper diaper 300, and are perpendicular to the up-down direction (direction perpendicular to the plane of the (paper diaper 300) in FIG. 1.

In the case of the present embodiment, the first diaper sensor 110 and the second diaper sensor 120 are configured similarly to each other, as one example.

The diaper sensor 100 (first diaper sensor 110 and second diaper sensor 120) is formed in a sheet shape. More specifically, the entirety of the diaper sensor 100 (first diaper sensor 110 and second diaper sensor 120) is formed in the shape of a flexible sheet.

The first diaper sensor 110 (second diaper sensor 120) is, for example, a stack formed in a flat plate shape. The planar shape of the first diaper sensor 110 (second diaper sensor 120) may be, but not specifically limited to, a substantially rectangular shape (e.g., rectangular shape with round corners) elongated in one direction as shown in FIG. 3, as one example.

In the case of the present embodiment, a mount component 50 is, as one example, an RFID chip (generally also called an IC chip), and the first diaper sensor 110 (second diaper sensor 120) is an RFID tag.

More specifically, as shown in FIGS. 1 to 3, the wetness detection sensor device 90 of each of the first diaper sensor 110 and the second diaper sensor 120 includes a board 10 having a base 11, a first conductive pattern 30 formed on the base 11, and the mount component 50 electrically connected to the first conductive pattern 30 and a second conductive pattern 40 forming the circuit 60 together with the first conductive pattern 30 in a complementary manner.

A portion of the base 11 corresponding to the second conductive pattern 40 is a missing portion 12 where the base 11 is missing. The second conductive pattern 40 is soluble in liquid, or is weakened due to contact with liquid.

Here, the portion of the base 11 corresponding to the second conductive pattern 40 is a portion corresponding to (overlapping with) the second conductive pattern 40 in plan view (as viewed in a direction perpendicular to the plane of the first diaper sensor 110 (second diaper sensor 120)).

Moreover, a phrase of the second conductive pattern 40 being soluble in liquid indicates that the second conductive pattern 40 is dissolved due to contact with liquid, and more specifically indicates that the second conductive pattern 40 is dissolved due to contact with water (moisture), for example. That is, the second conductive pattern 40 is, for example, soluble in water. It can be expected that the initial properties of the circuit 60 are no longer maintained in a state in which the second conductive pattern 40 is dissolved or weakened due to sufficient contact with liquid. For example, the state of the diaper sensor 100 being soaked in liquid can be detected by detection of disconnection of the circuit 60.

According to the present embodiment, since the diaper sensor 100 (first diaper sensor 110 and second diaper sensor 120) includes the board 10, the structural strength of the diaper sensor 100 can be sufficiently obtained. In addition, since the portion of the base 11 corresponding to the second conductive pattern 40 is the missing portion 12, liquid (urine) can easily contact the second conductive pattern 40 through the missing portion 12. Thus, the second conductive pattern 40 can be more reliably dissolved when the diaper sensor 100 is soaked in liquid. Consequently, both the structural strength of the diaper sensor 100 and reliability in detection of the state of the diaper sensor 100 being soaked in liquid can be favorably obtained.

Further, in the case of the present embodiment, the wetness detection sensor device 90 includes a soluble layer 21 arranged so as to face the base 11 and formed soluble in liquid or weakened when getting wet with liquid. The second conductive pattern 40 is formed on the other surface 21b of the soluble layer 21.

According to such a configuration, when slight external force is applied to the second conductive pattern 40 in a state in which the soluble layer 21 holding the second conductive pattern 40 is dissolved due to sufficient contact with liquid, it can be expected that the second conductive pattern 40 is easily broken or cracked and the initial properties of the circuit 60 are no longer maintained. That is, the state of the diaper sensor 100 being soaked in liquid can be more reliably sensed.

Note that the present invention is not limited to the above-described example and any one of the soluble layer 21 or the second conductive pattern 40 may be, for example, soluble in liquid.

In a case where only the soluble layer 21 of the soluble layer 21 and the second conductive pattern 40 is soluble in liquid, when slight external force is applied to the second conductive pattern 40 in a state in which the soluble layer 21 holding the second conductive pattern 40 is dissolved due to sufficient contact with liquid, it can be expected that the second conductive pattern 40 is easily broken or cracked and the initial properties of the circuit 60 are no longer maintained. A change (e.g., disconnection of the circuit 60) in the properties of the circuit 60 is detected so that the state of the first diaper sensor 110 (second diaper sensor 120) being soaked in liquid can be detected.

In a case where only the second conductive pattern 40 of the soluble layer 21 and the second conductive pattern 40 is soluble in liquid, it can be expected, as described above, that the second conductive pattern 40 is dissolved or weakened due to sufficient contact with liquid and the initial properties of the circuit 60 are no longer maintained. Thus, the state of the first diaper sensor 110 (second diaper sensor 120) being soaked in liquid can be detected.

As shown in FIGS. 1 and 2, in the wetness detection sensor device 90, the first conductive pattern 30 is directly stacked on one surface 11a of the base 11, and the second conductive pattern 40 is directly stacked on the first conductive pattern 30. Moreover, the soluble layer 21 is stacked on the base 11 through the first conductive pattern 30 and the second conductive pattern 40.

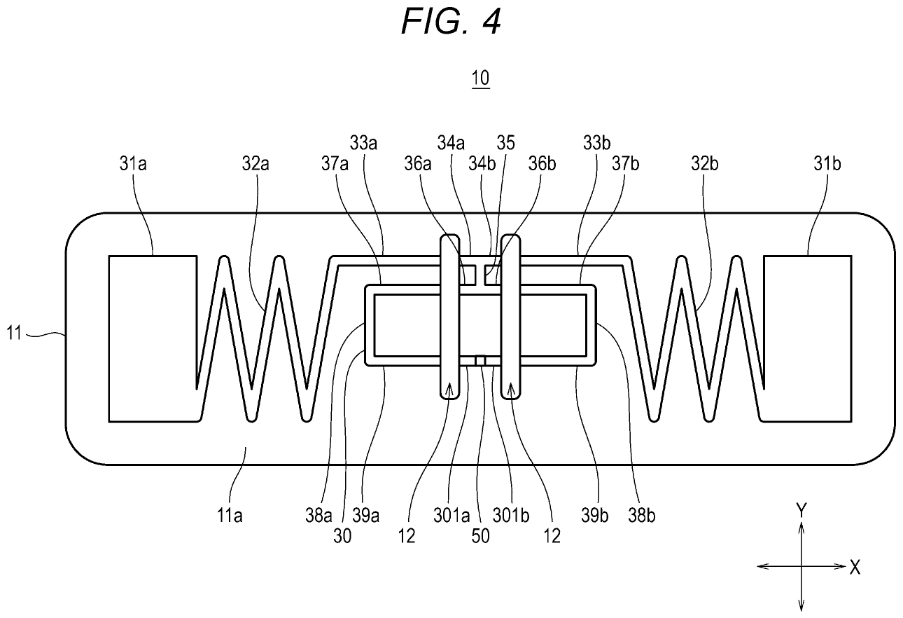

As shown in FIGS. 3 and 4, in the case of the present embodiment, the base 11 has, for example, multiple missing portions 12 each corresponding to later-described multiple second conductive patterns 40.

More specifically, the board 10 has, for example, a pair of right and left missing portions 12, and the right and left missing portions 12 have the same shape and are arranged symmetrically in the right-left direction. More specifically, the missing portion 12 is formed in a substantially rectangular shape (e.g., rectangular shape with round corners) elongated in the Y-direction, for example. Note that the shape of the missing portion 12 is not specifically limited.

Each missing portion 12 is an opening formed in the base 11. That is, each missing portion 12 penetrates the base 11 from the front to the back thereof. In a region where each missing portion 12 is present, no first conductive pattern 30 is formed. Each missing portion 12 may be in a slit shape.

In the case of the present embodiment, the first conductive pattern 30 has, as one example, an antenna wiring portion and a component mount wiring portion described below.

The antenna wiring portion includes a first antenna wiring portion located on the left side in FIG. 4 and a second antenna wiring portion located on the right side. The first antenna wiring portion has, for example, a wide portion 31a formed in a substantially rectangular shape elongated in the Y-direction in plan view and a zigzag portion 32a formed thinner than the wide portion 31a and extending zigzag in the Y-direction while extending in the X-direction. The left end of the zigzag portion 32a is connected to the wide portion 31a, and the right end of the zigzag portion 32a is connected to the component mount wiring portion. The second antenna wiring portion is formed, for example, symmetrical to the first antenna wiring portion in the right-left direction, and has a wide portion 31b and a zigzag portion 32b. The right end of the zigzag portion 32b is connected to the wide portion 31b, and the left end of the zigzag portion 32b is connected to the component mount wiring portion.

The component mount wiring portion has a linear pattern forming portion 33a connected to the right end of the zigzag portion 32a and extending in the X-direction, a linear pattern forming portion 34a arranged on the extension of the linear pattern forming portion 33a with the left missing portion 12 interposed therebetween and extending in the X-direction, a linear pattern forming portion 34b connected to the right side of the linear pattern forming portion 34a and extending in the X-direction, and a linear pattern forming portion 33b arranged on the extension of the linear pattern forming portion 34b with the right missing portion 12 interposed therebetween and extending in the X-direction.

The component mount wiring portion further has a coupling portion 35 and annular pattern forming portions 36a, 37a, 38a, 39a, 301a, 36b, 37b, 38b, 39b, 301b as described below.

One end of the coupling portion 35 is connected to the boundary between the linear pattern forming portion 34a and the linear pattern forming portion 34b, and the coupling portion 35 extends to one side (below in FIG. 4) from such a boundary in the Y-direction (i.e., extends in the Y-direction).

The annular pattern forming portion 36a is connected to the other end of the coupling portion 35, and extends leftward from the other end of the coupling portion 35 (i.e., extends in the X-direction). The annular pattern forming portion 37a is arranged on the extension of the annular pattern forming portion 36a with the left missing portion 12 interposed therebetween, and extends in the X-direction. One end of the annular pattern forming portion 38a is connected to the left end of the annular pattern forming portion 37a, and the annular pattern forming portion 38a extends to one side (below in FIG. 4) from the left end of the annular pattern forming portion 37a in the Y-direction (i.e., extends in the Y-direction). The annular pattern forming portion 39a is connected to the other end of the annular pattern forming portion 38a, and extends rightward from the other end of the annular pattern forming portion 38a (i.e., extends in the X-direction). The annular pattern forming portion 301a is arranged on the extension of the annular pattern forming portion 39a with the left missing portion 12 interposed therebetween, and extends in the X-direction.

The annular pattern forming portion 36b is connected to the other end of the coupling portion 35, and extends rightward from the other end of the coupling portion 35 (i.e., extends in the X-direction). The annular pattern forming portion 37b is arranged on the extension of the annular pattern forming portion 36b with the right missing portion 12 interposed therebetween, and extends in the X-direction. One end of the annular pattern forming portion 38b is connected to the right end of the annular pattern forming portion 37b, and the annular pattern forming portion 38b extends to one side (below in FIG. 4) from the right end of the annular pattern forming portion 37b in the Y-direction (i.e., extends in the Y-direction). The annular pattern forming portion 39b is connected to the other end of the annular pattern forming portion 38b, and extends leftward from the other end of the annular pattern forming portion 38b (i.e., extends in the X-direction). The annular pattern forming portion 301b is arranged on the extension of the annular pattern forming portion 39b with the right missing portion 12 interposed therebetween, and extends in the X-direction.

The annular pattern forming portion 301a is arranged on the extension of the annular pattern forming portion 301b, and vice versa.

The soluble layer 21 contains polyvinyl alcohol (PVA), for example. Since the soluble layer 21 contains PVA, a structure of the soluble layer 21 being favorably dissolved when the diaper sensor 100 contacts moisture can be achieved.

Note that in a case where the soluble layer 21 is soluble in water, the material of the soluble layer 21 is not limited to PVA and may be, for example, a polyvinylpyrrolidone, water-soluble polyester, or water-soluble paper material or a sheet material derived from a starch component.

Further, in the case of the present embodiment, the soluble layer 21 contains resin having hot melt properties, and is hot-melt joined to each of the base 11 and the first conductive pattern 30. That is, the soluble layer 21 is fusion-joined to each of the base 11 and the first conductive pattern 30.

Note that the present invention is not limited to such an example and the soluble layer 21 may be joined to each of the base 11 and the first conductive pattern 30, for example, with a water-soluble adhesive (not shown).

The second conductive pattern 40 is formed, for example, on the other surface 21b side of the soluble layer 21.

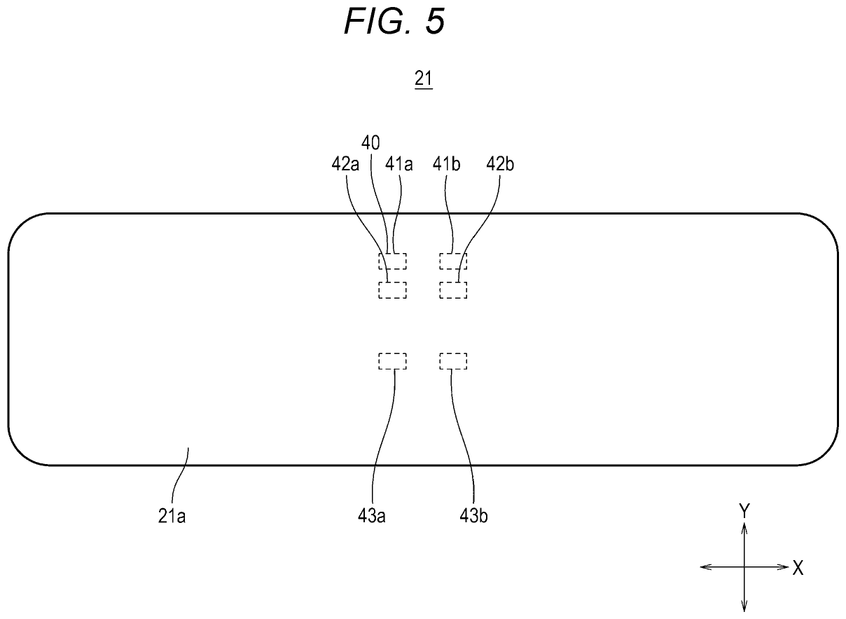

As shown in FIG. 5, the diaper sensor 100 has, as the second conductive pattern 40, bridge portions 41a, 41b, 42a, 42b, 43a, 43b extending in the X-direction, for example. That is, the diaper sensor 100 has, for example, multiple second conductive patterns 40.

As shown in FIG. 3, the bridge portion 41a connects a right end portion of the linear pattern forming portion 33a and a left end portion of the linear pattern forming portion 34a to each other. The bridge portion 41b connects a right end portion of the linear pattern forming portion 34b and a left end portion of the linear pattern forming portion 33b to each other. With this configuration, the linear pattern forming portion 33a, the bridge portion 41a, the linear pattern forming portion 34a, the linear pattern forming portion 34b, the bridge portion 41b, and the linear pattern forming portion 33b form a linear pattern linearly extending in the X-direction and connecting the first antenna wiring portion and the second antenna wiring portion to each other.

The bridge portion 42a connects a right end portion of the annular pattern forming portion 37a and a left end portion of the annular pattern forming portion 36a to each other. The bridge portion 42b connects a right end portion of the annular pattern forming portion 36b and a left end portion of the annular pattern forming portion 37b to each other. The bridge portion 43a connects a right end portion of the annular pattern forming portion 39a and a left end portion of the annular pattern forming portion 301a to each other. The bridge portion 43b connects a right end portion of the annular pattern forming portion 301b and a left end portion of the annular pattern forming portion 39b to each other. With this configuration, the annular pattern forming portion 301a, the bridge portion 43a, the annular pattern forming portion 39a, the annular pattern forming portion 38a, the annular pattern forming portion 37a, the bridge portion 42a, the annular pattern forming portion 36a, the annular pattern forming portion 36b, the bridge portion 42b, the annular pattern forming portion 37b, the annular pattern forming portion 38b, the annular pattern forming portion 39b, the bridge portion 43b, and the annular pattern forming portion 301b form an annular pattern. The annular pattern is formed, for example, in a substantially rectangular annular shape in plan view. The annular pattern is non-continuous between the annular pattern forming portion 301a and the annular pattern forming portion 301b. That is, the annular pattern is in a shape (opened annular shape) having an opening.

The coupling portion 35 couples the linear pattern and the annular pattern to each other.

Each of the bridge portion 41a, the bridge portion 42a, and the bridge portion 43a is arranged over the left missing portion 12, and each of the bridge portion 41b, the bridge portion 42b, and the bridge portion 43b is arranged over the right missing portion 12.

As shown in FIG. 1, the bridge portion 43a is bridged between the annular pattern forming portion 39a and the annular pattern forming portion 301a to electrically connect the annular pattern forming portion 39a and the annular pattern forming portion 301a to each other. Similarly, the bridge portion 43b is bridged between the annular pattern forming portion 301b and the annular pattern forming portion 39b to electrically connect the annular pattern forming portion 301b and the annular pattern forming portion 39b to each other.

Similarly, the bridge portion 41a is bridged between the linear pattern forming portion 33a and the linear pattern forming portion 34a to electrically connect the linear pattern forming portion 33a and the linear pattern forming portion 34a to each other. The bridge portion 41b is bridged between the linear pattern forming portion 34b and the linear pattern forming portion 33b to electrically connect the linear pattern forming portion 34b and the linear pattern forming portion 33b to each other. The bridge portion 42a is bridged between the annular pattern forming portion 37a and the annular pattern forming portion 36a to electrically connect the annular pattern forming portion 37a and the annular pattern forming portion 36a to each other. The bridge portion 42b is bridged between the annular pattern forming portion 36b and the annular pattern forming portion 37b to electrically connect the annular pattern forming portion 36b and the annular pattern forming portion 37b to each other.

As described above, the second conductive pattern 40 (each of the bridge portions 41a, 41b, 42a, 42b, 43a, 43b) is arranged over the missing portion 12, and is bridged between a first portion of the first conductive pattern 30 and a second portion of the first conductive pattern 30 separated from the first portion through the missing portion 12 to electrically connect the first portion and the second portion to each other. That is, each second conductive patter 40 is a bridge wiring. The second conductive pattern 40 extends, in plan view, across the missing portion 12 from part of a peripheral edge portion of the missing portion 12 to another portion of the peripheral edge portion of the missing portion 12.

Note that the diaper sensor 100 may have, as the second conductive pattern 40, a portion other than the above-described bridge portions 41a to 43b and the bridge portions 41a to 43b are wiring portions forming the circuit 60.

That is, a portion (bridge portions 41a to 43b) of the second conductive pattern 40 corresponding to the missing portion 12 is not, e.g., an electrode, but a wiring portion forming the circuit 60.

The second conductive pattern 40 (each of the bridge portions 41a, 41b, 42a, 42b, 43a, 43b) is preferably formed thicker than a portion (linear pattern forming portions 33a, 33b, 34a, 34b, 36a, 36b, 37a, 37b, 39a, 39b, 301a, 301b) of the first conductive pattern 30 connected to the second conductive pattern 40. With this configuration, when the board 10 and the soluble layer 21 are assembled with aligned with each other and the second conductive pattern 40 and the first conductive pattern 30 are electrically connected to each other, even if the board 10 and the soluble layer 21 are slightly relatively misaligned in the planar direction, the second conductive pattern 40 can be connected to the first conductive pattern 30 with properly aligned with the first conductive pattern 30. Note that in a case where the first conductive pattern 30 is, for example, a pattern derived from metal foil and the impedance thereof is low, if the second conductive pattern 40 is a coating film formed by printing, the impedance of the second conductive pattern 40 is expected to be a higher value than the impedance of the above-described pattern made of metal foil. However, the second conductive pattern 40 is formed thicker than the portion of the first conductive pattern 30 connected to the second conductive pattern 40 as described above, and it is advantageous because impedance matching between the first conductive pattern 30 and the second conductive pattern 40 can be made in a low impedance state.

Note that the second conductive pattern 40 may be formed with the same width as that of the portion of the first conductive pattern 30 connected to the second conductive pattern 40 or be formed thinner than such a portion.

The length of each second conductive pattern 40 is preferably longer than the width dimension of the portion (linear pattern forming portions 33a. 33b, 34a, 34b. 36a, 36b, 37a, 37b. 39a, 39b, 301a, 301b) of the first conductive pattern 30 connected to the second conductive pattern 40, more preferably a length twice as long as the width dimension or more, and much more preferably a length three times as long as the width dimension or more.

Specifically, the length (bridge length) of a portion of each second conductive pattern 40 bridged over the first conductive pattern 30 (between the first portion and the second portion) is preferably longer than the width dimension of the portion of the first conductive pattern 30 connected to the second conductive pattern 40, more preferably a length twice as long as the width dimension or more, and much more preferably a length three times as long as the width dimension or more.

The second conductive pattern 40 is, for example, a water-soluble silver pattern.

The thickness dimension of the second conductive pattern 40 is, but not specifically limited to, preferably 10 μm or more and 50 μm or less, for example. Since the thickness dimension of the second conductive pattern 40 is 10 μm or more, the stable properties of the circuit 60 can be obtained at a stage before contact of the first diaper sensor 110 (second diaper sensor 120) with liquid. Since the thickness dimension of the second conductive pattern 40 is 50 μm or less, the second conductive pattern 40 can be quickly dissolved or weakened when the first diaper sensor 110 (second diaper sensor 120) contacts liquid.

In the case of the present embodiment, since the second conductive patterns 40 as the bridge wirings are arranged at multiple locations on the circuit 60, a probability of any of the second conductive patterns 40 being broken, cracked, or dissolved or weakened upon contact of the first diaper sensor 110 (second diaper sensor 120) with liquid can be increased.

In the case of the present embodiment, the base 11 is not soluble in liquid. Thus, even when the first diaper sensor 110 (second diaper sensor 120) accidentally contacts liquid upon, e.g., storage, a probability of the properties of the circuit 60 being changed due to breakage or cracking of the first conductive pattern 30 by dissolving of the base 11 can be reduced.

The base 11 insoluble in liquid may be made of paper or a resin film, for example. Examples of a resin material forming the resin film may include, but not specifically limited to, polyethylene terephthalate, polyethylene, polystyrene, polypropylene, and polyester.

Note that the present invention is not limited to such an example and the base 11 may be soluble in liquid.

The thickness dimension of the base 11 is, but not specifically limited to, preferably 10 μm or more and 100 μm or less, for example. Since the thickness dimension of the base 11 is 10 μm or more, the base 11 can stably support the first conductive pattern 30 and the structural strength of the first diaper sensor 110 (second diaper sensor 120) can be sufficiently obtained.

Since the thickness dimension of the base 11 is 100 μm or less, favorable flexibility of the first diaper sensor 110 (second diaper sensor 120) is obtained.

In the case of the present embodiment, the first conductive pattern 30 is also not soluble in liquid. Thus, a probability of the properties of the circuit 60 being changed due to dissolving of the first conductive pattern 30 even when the first diaper sensor 110 (second diaper sensor 120) accidentally contacts liquid upon, e.g., storage can be reduced. Specifically, over the half of the circuit 60 is preferably formed by the first conductive pattern 30 insoluble in liquid.

Note that the present invention is not limited to such an example and the first conductive pattern 30 may be soluble in liquid.

The first conductive pattern 30 is, for example, a coating film formed by printing or metal foil.

The first conductive pattern 30 is, for example, a coating film containing a conductive filler and a binder containing thermoplastic resin. Examples of the conductive filler may include gold, silver, copper, and carbon. Examples of the thermoplastic resin may include polyester resin, acrylic resin, and urethane resin.

The first conductive pattern 30 made of metal foil may be formed, for example, by punching or etching.

The thickness dimension of the first conductive pattern 30 is, but not specifically limited to, preferably 5 μm or more and 30 μm or less, for example.

Note that the outer shape of the board 10 (base 11) and the outer shape of the soluble layer 21 may be the same as each other or be different from each other. For example, in the case of the present embodiment, the outer shape of the board 10 (base 11) and the outer shape of the soluble layer 21 are the same as each other, and are coincident with each other in plan view.

Although not shown in detail, the mount component 50 includes, for example, a component body having an element inside by resin molding and a mount terminal provided along the lower surface of the component body. The element and the mount terminal are electrically connected to each other in the resin-molded body.

The number of mount terminals included in the mount component 50 is not specifically limited. In the case of the present embodiment, the mount component 50 includes two mount terminals, and each mount terminal is electrically connected to the first conductive pattern 30. More specifically, one mount terminal of the mount component 50 is electrically connected to a right end portion of the annular pattern forming portion 301a, and the other mount terminal of the mount component 50 is electrically connected to a left end portion of the annular pattern forming portion 301b. That is, the mount component 50 is arranged over the annular pattern forming portion 301a and the annular pattern forming portion 301b.

As described above, the mount component 50 is mounted on the component mount wiring portion.

The antenna wiring portion exchanges a signal with not-shown external equipment (e.g., RFID reader/writer), for example. A signal or radio wave received from the external equipment by the antenna wiring portion is input to the mount component 50. The mount component 50 transmits a signal to the external equipment via the component mount wiring portion and the antenna wiring portion. Note that in some cases, part or the entirety of the component mount wiring portion may also function as an antenna in cooperation with the antenna wiring portion.

The mount component 50 is, for example, of a passive type to be operated with power excited from the external equipment via the antenna wiring portion.

In the case of the present embodiment, the communication function of the first diaper sensor 110 (second diaper sensor 120) is eliminated or degraded by dissolving (or weakening) of the second conductive pattern 40 due to contact of the first diaper sensor 110 (second diaper sensor 120) with liquid. Elimination or degradation of the communication function is detected by the external equipment so that contact of the first diaper sensor 110 (second diaper sensor 120) with liquid can be detected.

As shown in FIGS. 1 and 2, in the case of the present embodiment, the diaper sensor 100 (first diaper sensor 110 and second diaper sensor 120) includes both the first water-absorbing sheet 81 and the second water-absorbing sheet 83.

With this configuration, liquid (urine) can be absorbed by each of the first water-absorbing sheet 81 and the second water-absorbing sheet 83, and therefore, the liquid can be sufficiently supplied to the second conductive pattern 40 and the soluble layer 21. In addition, seepage of the second conductive pattern 40 and soluble layer 21 dissolved in urine or liquid can be reduced by each of the first water-absorbing sheet 81 and the second water-absorbing sheet 83, and therefore, a user's uncomfortable feeling can be reduced.

More specifically, in the first diaper sensor 110, the urine of the user can be sufficiently supplied to the second conductive pattern 40 and the soluble layer 21 by the first water-absorbing sheet 81.

In the second diaper sensor 120, urine having leaked from an absorber 350 to the diaper body 300a can be sufficiently supplied to the second conductive pattern 40 and the soluble layer 21 by the second water-absorbing sheet 83, and seepage of the second conductive pattern 40 and soluble layer 22 dissolved in urine or liquid to the user side can be reduced by the first water-absorbing sheet 81.

Further, the urine of the user can be quickly supplied to the first water-absorbing sheet 81 and therefore to the second conductive pattern 40 and the soluble layer 21 by the highly water-permeable sheet 85. Thus, a feeling of smoothness of the surface (highly water-permeable sheet 85) of the diaper sensor 100 on the user's body surface side can be maintained, and therefore, a user's uncomfortable feeling can be reduced.

In the case of the present embodiment, the first water-absorbing sheet 81 is directly stacked on one surface (one surface 21a) of the wetness detection sensor device 90 and the second water-absorbing sheet 83 is directly arranged on the other surface (other surface 11b) of the wetness detection sensor device 90, as one example. Moreover, the highly water-permeable sheet 85 is directly stacked on the first water-absorbing sheet 81.

As described above, each of the first water-absorbing sheet 81 and the second water-absorbing sheet 83 is higher in water absorption properties (has better water absorption properties) than the highly water-permeable sheet 85.

More specifically, the absorbency of each of the first water-absorbing sheet 81 and the second water-absorbing sheet 83 is higher than the absorbency of the highly water-permeable sheet 85, as one example.

Here, the “absorbency” can be evaluated, for example, by a testing method defined in JIS L 1907 (testing methods for water absorbency of textiles). First, in such a testing method, in a state in which non-woven fabric cut out into a sufficient length with a fiber direction as a longitudinal direction is hung, a tip end of the non-woven fabric is dipped in colored water for 60 seconds, and then, is left for 30 minutes. Thereafter, the level of water having elevated (absorbed) in the non-woven fabric in the longitudinal direction thereof is evaluated.

Each of the first water-absorbing sheet 81 and the second water-absorbing sheet 83 is, but not specifically limited to, high water-absorption (water-holding) non-woven fabric such as pulp, cotton, hemp, or rayon, as one example. As one example, the absorbency of each of the first water-absorbing sheet 81 and the second water-absorbing sheet 83 is preferably 100 mm or more and 200 mm or less and more preferably 110 mm or more and 180 mm or less.

The highly water-permeable sheet 85 is, but not specifically limited to, non-woven fabric made of polyester, polypropylene, or polyethylene terephthalate and having a basis weight of 5 g/m2 or more and 100 g/m2 or less and preferably 10 g/m2 or more and 50 g/m2 or less, as one example. With this configuration, the water permeability of the highly water-permeable sheet 85 can be favorably ensured.

As described above, each of the first diaper sensor 110 and the second diaper sensor 120 is provided on the paper diaper 300 (see FIG. 6). Note that FIG. 6 shows the surface of the paper diaper 300 on the side of contact with the skin of the user.

The paper diaper body 301 includes, for example, a back portion 310 arranged on the back side of the user, a front portion 320 arranged on the belly side of the user, a coupling portion 330 coupling the back portion 310 and the front portion 320 to each other, and multiple hook-and-loop fastener portions 340 formed at side portions of the back portion 310, as shown in FIG. 6. Both side portions of the back portion 310 and both side portions of the front portion 320 are coupled to each other through the hook-and-loop fastener portions 340 so that the user can wear the paper diaper 300.

The absorber 350 capable of absorbing liquid is formed on the surface of the paper diaper 300 on the side of contact with the skin of the user.

The absorber 350 extends, for example, in the front-back direction over the back portion 310, the front portion 320, and the coupling portion 330.

In a state in which the user wears the paper diaper 300, the absorber 350 is arranged at a location where the absorber 350 contacts the external urethral opening of the user. When the user urinates, the urine is absorbed by the absorber 350.

Each of the first diaper sensor 110 and the second diaper sensor 120 is preferably arranged, for example, on the surface of the paper diaper body 301 on the side of contact with the skin of the user.

In the case of the present embodiment, each of the first diaper sensor 110 and the second diaper sensor 120 is bonded, as one example, to the paper diaper body 301 through a double-sided tape 88 or a hook-and-loop fastener (not shown).

Moreover, each of the first diaper sensor 110 and the second diaper sensor 120 is attached to the paper diaper body 301 in such a posture that one surface (one surface 21a) side of the wetness detection sensor device 90 is arranged on the side of contact with the skin of the user and the other surface (other surface 11b) side of the wetness detection sensor device 90 is arranged on the side (i.e., diaper body 301 side) opposite to the side of contact with the skin of the user. In the example shown in FIGS. 1 and 2, the second water-absorbing sheet 83 is attached, through the double-sided tape 88, to the surface of the paper diaper body 301 on the side of contact with the skin of the user.

The first diaper sensor 110 is provided, for example, at the absorber 350 of the paper diaper body 301. The first diaper sensor 110 may be provided at a portion of the absorber 350 corresponding to the coupling portion 330 or a portion of the absorber 350 corresponding to the front portion 320 as long as the first diaper sensor 110 is within the area of the portion 302 corresponding to the external urethral opening. That is, the arrangement location of the first diaper sensor 110 is changeable as necessary within the area of the portion 302 corresponding to the external urethral opening, and therefore, the first diaper sensor 110 is applicable regardless of the gender of the user.

In the example shown in FIG. 6, the first diaper sensor 110 is arranged in such a posture that the longitudinal direction of the first diaper sensor 110 is the front-back direction.

The second diaper sensor 120 is preferably arranged, for example, along the peripheral edge (e.g., upper edge 310a of the back portion 310 or lower edge of the front portion 320) of a portion of the paper diaper body 301 other than the portion 302 corresponding to the external urethral opening.

In the case of the present embodiment, the second diaper sensor 120 is arranged, for example, at an outer peripheral portion (e.g., back side) of the paper diaper body 301.

With this configuration, urine having leaked out of the paper diaper body 301 can be more reliably detected by the second diaper sensor 120.

More specifically, the second diaper sensor 120 is provided, for example, along the upper edge 310a of the back portion 310 arranged on the back side of the user. Moreover, the second diaper sensor 120 is arranged, for example, in such a posture that the longitudinal direction of the second diaper sensor 120 is the right-left direction.

Note that in the present invention, the shape, orientation, arrangement, posture, or the like of each of the first diaper sensor 110 and the second diaper sensor 120 is not specifically limited and, e.g., the size or use application of the paper diaper body 301 may be set as necessary.

More specifically, each of the first diaper sensor 110 and the second diaper sensor 120 may be arranged, for example, in such a posture that one surface (one surface 21a) of the wetness detection sensor device is on the paper diaper body 301 side and the other surface (other surface 11b) of the wetness detection sensor device is on the side of contact with the skin of the user. The second diaper sensor 120 may be formed in a band shape, and may be provided along the upper edge 310a of the back portion 310, for example. In this case, the second conductive patterns 40 are preferably provided at multiple locations along the longitudinal direction of the second diaper sensor 120, for example.

In the present invention, each of the number of first diaper sensors 110 and the number of second diaper sensors 120 on the paper diaper 300 is not specifically limited. Each of the number of first diaper sensors 110 and the number of second diaper sensors 120 on the paper diaper 300 may be two or more.

In this case, multiple first diaper sensors 110 may be arranged, for example, side by side in the front-back direction at the portion 302 corresponding to the external urethral opening. Moreover, multiple second diaper sensors 120 may be arranged, for example, side by side along the upper edge 310a of the back portion 310.

The present invention is not limited to the above-described example, and for example, may be a paper diaper kit including the paper diaper 300 and an incontinence pad (not shown).

In this case, the incontinence pad has an incontinence pad body (not shown) and a pad-side sensor device provided on the incontinence pad body, and the pad-side sensor device is the diaper sensor 100.

The paper diaper 300 includes the paper diaper body 301 and the diaper sensor 100 provided at a portion of the paper diaper body 301 different from the portion 302 corresponding to the external urethral opening.

The incontinence pad has, for example, an adhesive layer for attaching the incontinence pad to the paper diaper body 301, and is used with attached to the paper diaper body 301.

According to such a configuration, when the user urinates in the incontinence pad, urination of the user can be detected by the pad-side sensor device. In a case where the urine of the user has leaked out of the incontinence pad, such urine leakage can be detected by the diaper sensor 100. That is, urination of the user and the urine leakage from the incontinence pad can be separately detected.

Next, a method for manufacturing the first diaper sensor 110 according to the present embodiment will be described.

The method for manufacturing the first diaper sensor 110 in the present embodiment includes a step of preparing the board 10, a step of preparing the soluble layer 21 formed with the second conductive pattern 40, and a step of assembling the board 10 and the soluble layer 21 to each other.

In the step of preparing the board 10, the board 10 (see FIG. 4) having the base 11, the first conductive pattern 30 formed on the base 11, and the mount component 50 electrically connected to the first conductive pattern 30 is prepared.

In the step of preparing the soluble layer 21, the second conductive pattern 40 is formed, by printing, on the other surface 21b of the soluble layer 21.

In the step of assembling the board 10 and the soluble layer 21 to each other, the board 10 and the soluble layer 21 are assembled to each other such that the first conductive pattern 30 and the second conductive pattern 40 form the circuit 60 in a complementary manner and the portion of the base 11 corresponding to the second conductive pattern 40 forms the missing portion 12 where the base 11 is missing. That is, the board 10 and the soluble layer 21 are aligned with facing each other, and then, are bonded to each other.

In this manner, the first diaper sensor 110 in the present embodiment is obtained. The second diaper sensor 120 is also obtained by a method similar to the above-described manufacturing method.

The board 10 and the soluble layer 21 can be bonded in such a manner that the board 10 and the soluble layer 21 are thermocompressed (thermally laminated) to each other. As one example, a step of thermocompressing the board 10 and the soluble layer 21 to each other is performed preferably at 80° C. or more and 150° C. or less and more preferably 100° C. or more and 120° C. or less. A heating time may be, for example, about 5 to 10 seconds. Moreover, the thermocompressing step is preferably performed under a pressurization condition of 0.3 MPa or more and 3.0 MPa or less and more preferably a pressurization condition of 1.0 MPa or more and 2.0 MPa or less.

The board 10 and the soluble layer 21 are bonded as described above so that the second conductive pattern 40 can be favorably electrically connected to the first conductive pattern 30.

A technique of forming the missing portion 12 in the board 10 is not specifically limited, and as one representative example, the missing portion 12 may be formed by punching and a blade or a punch may be mainly applied. Note that the present invention is not limited to such an example and, e.g., laser machining is also applicable.

### Modifications

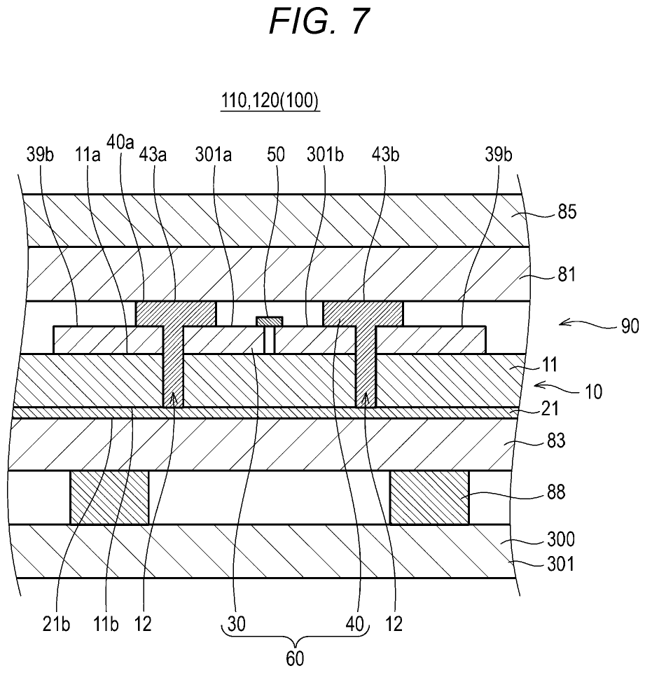

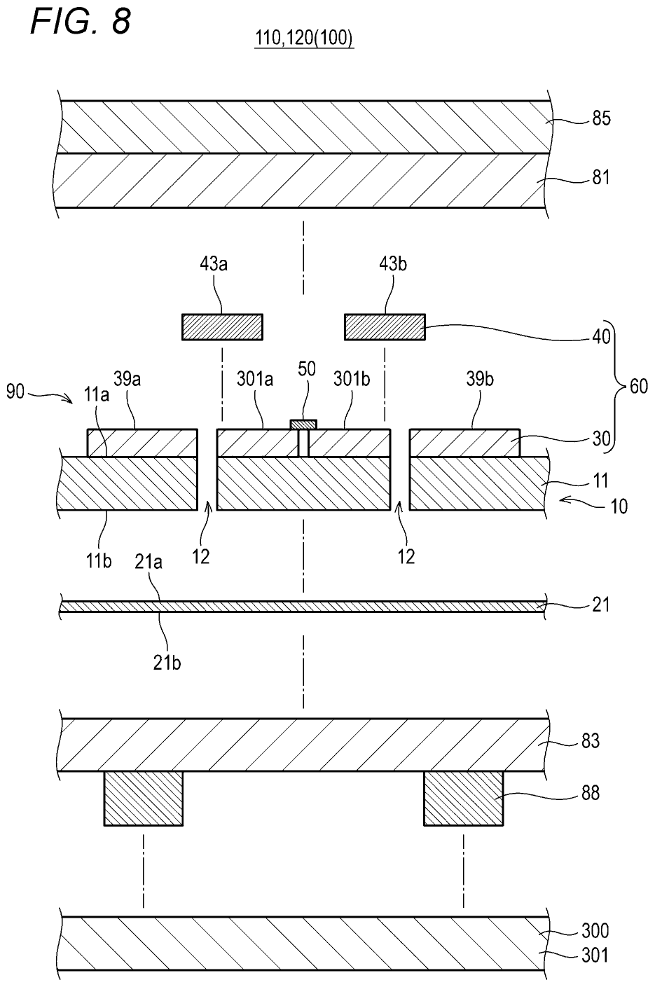

Next, a modification will be described using FIGS. 7 and 8.

A diaper sensor 100 according to the present embodiment is different from the diaper sensor 100 according to the above-described embodiment on points described below, and is configured similarly to the diaper sensor 100 according to the above-described embodiment on the other points.

In the above-described embodiment, the example where the soluble layer 21 is stacked on the base 11 through the first conductive pattern 30 and the second conductive pattern 40 has been described, but in the case of the present modification, the base 11 is directly stacked on the soluble layer 21 as shown in FIGS. 7 and 8.

More specifically, in the case of the present modification, in the wetness detection sensor device 90, the soluble layer 21 is arranged on the other surface 11b of the base 11, and part of the second conductive pattern 40 is stacked on the first conductive pattern 30. In other words, the board 10 including the base 11 and the first conductive pattern 30 is arranged, in the up-down direction, between the part of the second conductive pattern 40 and the soluble layer 21.

That is, in the case of the present modification, the surface of the wetness detection sensor device 90 on the side of contact with the skin of the wearer is formed by one surface 40a of the second conductive pattern 40, and the surface of the wetness detection sensor device 90 on the side (i.e., diaper body 301 side) opposite to the side of contact with the skin of the user is formed by the other surface 21b of the soluble layer 21.

According to such a configuration, urine having leaked out of the paper diaper body 301 can easily contact the soluble layer 21 and therefore the circuit 60. Thus, the urine leakage can be more reliably detected by the diaper sensor 100.

More specifically, in the case of the present modification, a portion of the board 10 corresponding to the second conductive pattern 40 is also a missing portion 12 as in the above-described embodiment. Part of the second conductive pattern 40 enters the missing portion 12, and is connected to one surface 21a of the soluble layer 21 through the missing portion 12. With this configuration, the second conductive pattern 40 and the soluble layer 21 are connected to each other with the board 10 interposed therebetween.

In the case of the present modification, when the second conductive pattern 40 is formed, the board 10 is first directly stacked on one surface 21a of the soluble layer 21. Then, the second conductive pattern 40 is formed, by printing, on the first conductive pattern 30 and inside the missing portion 12.

The embodiments have been described above with reference to the drawings. These embodiments are examples of the present invention, and various configurations other than those described above can also be employed.

For example, the example where the first diaper sensor 110 and the second wetness detection device are configured similarly to each other has been described above, but the present invention is not limited to such an example and the first diaper sensor 110 and the second wetness detection device may have different configurations.

In this case, at least one of the first diaper sensor 110 or the second diaper sensor 120 has multiple second conductive patterns, and the base 11 has multiple missing portions 12 each corresponding to the multiple second conductive patterns 40.

The example where the diaper sensor 100 includes both the first water-absorbing sheet 81 and the second water-absorbing sheet 83 has been described above, but the present invention is not limited to such an example and the diaper sensor 100 may include only any one of the first water-absorbing sheet 81 or the second water-absorbing sheet 83.

In this case, the first diaper sensor 110 preferably includes, for example, the highly water-permeable sheet 85 and the first water-absorbing sheet 81 of the first water-absorbing sheet 81 interposed between the highly water-permeable sheet 85 and the wetness detection sensor device 90 and the second water-absorbing sheet 83 arranged on the opposite side of the wetness detection sensor device 90 from the highly water-permeable sheet 85 side.

With this configuration, the urine of the user can be more easily supplied to the circuit 60 through the highly water-permeable sheet 85 and the first water-absorbing sheet 81.

For example, the second diaper sensor 120 preferably includes the highly water-permeable sheet 85 and the second water-absorbing sheet 83 of the first water-absorbing sheet 81 interposed between the highly water-permeable sheet 85 and the wetness detection sensor device 90 and the second water-absorbing sheet 83 arranged on the opposite side of the wetness detection sensor device 90 from the highly water-permeable sheet 85 side.

With this configuration, urine having leaked out of the paper diaper body 301 side can be favorably absorbed by the second water-absorbing sheet 83, and therefore, can be more easily supplied to the circuit 60.

The example where the first water-absorbing sheet 81 is directly stacked on one surface of the wetness detection sensor device 90 has been described above, but the present invention is not limited to such an example. The diaper sensor 100 may have a sheet-shaped spacer (not shown) interposed between the first water-absorbing sheet 81 and one surface of the wetness detection sensor device 90, and a portion of the spacer corresponding to the second conductive pattern 40 may be a missing portion where the spacer is missing.

Similarly, the example where the other surface of the wetness detection sensor device 90 is directly stacked on the second water-absorbing sheet 83 has been described above, but the present invention is not limited to such an example. The diaper sensor 100 may have a sheet-shaped spacer (not shown) interposed between the second water-absorbing sheet 83 and the other surface of the wetness detection sensor device 90, and a portion of the spacer corresponding to the second conductive pattern 40 may be a missing portion where the spacer is missing.

The example where the mount component 50 is the RFID chip has been described above, but the present invention is not limited to such an example. The mount component 50 may be other electronic components such as a capacitor or a resistor.

The example where the second conductive pattern 40 is bridged between the first portion of the first conductive pattern 30 and the second portion of the first conductive pattern 30 separated from the first portion through the missing portion 12 has been described above, but the present invention is not limited to such an example. The second conductive pattern 40 is not necessarily bridged over the first conductive pattern 30, but may be merely connected to the first conductive pattern 30. As one simple example, the left half of the circuit 60 shown in FIG. 1 is formed by the first conductive pattern 30, and the right half is formed by the second conductive pattern 40.

The example where the board 10 has the two missing portions 12 has been described above, but the number of missing portions 12 formed in the board 10 is not specifically limited and may be three or more, for example.

The present embodiments include the following technical ideas.
(1) A diaper sensor including a sheet-shaped wetness detection sensor device having a circuit breakable or damageable when getting wet with liquid, including: a highly water-permeable sheet forming a surface of the diaper sensor arranged on a user's body surface side; and at least any one of a first water-absorbing sheet interposed between the highly water-permeable sheet and the wetness detection sensor device or a second water-absorbing sheet arranged on an opposite side of the wetness detection sensor device from a highly water-permeable sheet side.
(2) The diaper sensor according to (1), in which both the first water-absorbing sheet and the second water-absorbing sheet are included in the diaper sensor.
(3) The diaper sensor according to (1) or (2), in which the wetness detection sensor device includes a board having a base, a first conductive pattern formed on the base, and a mount component electrically connected to the first conductive pattern, and a second conductive pattern forming the circuit together with the first conductive pattern in a complementary manner, a portion of the base corresponding to the second conductive pattern is a missing portion where the base is missing, and the second conductive pattern is soluble in liquid, or is weakened when getting wet with liquid.
(4) The diaper sensor according to (3), in which the second conductive pattern includes multiple second conductive patterns, and the missing portion of the base includes multiple missing portions each corresponding to the multiple second conductive patterns.
(5) The diaper sensor according to (1) to (4), in which the diaper sensor is formed in a sheet shape.
(6) A paper diaper including: a paper diaper body; a first diaper sensor provided at a portion of the paper diaper body corresponding to an external urethral opening; and a second diaper sensor provided at a portion of the paper diaper body different from that for the first diaper sensor, in which each of the first diaper sensor and the second diaper sensor is the diaper sensor according to any one of (1) to (5).
(7) The paper diaper according to (6), in which the second diaper sensor is arranged at a peripheral edge portion of the paper diaper body.
(8) A paper diaper including: a paper diaper body; and a diaper sensor provided at a portion of the paper diaper body different from a portion corresponding to an external urethral opening, the diaper sensor being the diaper sensor according to any one of (1) to (5).
(9) A paper diaper kit including: the paper diaper according to (8); and an incontinence pad, the incontinence pad has an incontinence pad body and a pad-side sensor device provided on the incontinence pad body, and the pad-side sensor device is the diaper sensor according to any one of (1) to (5).

### LIST OF REFERENCE SIGNS

10 Board
11 Base
11a One Surface
11b Other Surface
12 Missing Portion
21 Soluble Layer
21a One Surface
21b Other Surface
30 First Conductive Pattern
30a, 30b End
31a, 31b Wide Portion
32a, 32b Zigzag Portion
33a, 33b, 34a, 34b Linear Pattern Forming Portion
35 Coupling Portion
36a, 36b, 37a, 37b, 38a, 38b, 39a, 39b, 301a, 301b Annular Pattern Forming Portion
40 Second Conductive Pattern
41a, 41b, 42a, 42b, 43a, 43b Bridge Portion
50 Mount Component
60 Circuit
81 First Water-Absorbing Sheet
83 Second Water-Absorbing Sheet
85 Highly Water-Permeable Sheet
88 Double-Sided Tape
90 Wetness Detection Sensor Device
100 Diaper Sensor
110 First Diaper Sensor
120 Second Diaper Sensor
300 Paper Diaper
301 Paper Diaper Body
302 Portion Corresponding to External Urethral Opening
310 Back Portion
310a Upper Edge
320 Front Portion
330 Coupling Portion
340 Hook-And-Loop Fastener Portion
350 Absorber

1. A diaper sensor including a sheet-shaped wetness detection sensor device having a circuit breakable or damageable when getting wet with liquid, comprising:
a highly water-permeable sheet forming a surface of the diaper sensor arranged on a user's body surface side; and
at least any one of a first water-absorbing sheet interposed between the highly water-permeable sheet and the wetness detection sensor device or a second water-absorbing sheet arranged on an opposite side of the wetness detection sensor device from a highly water-permeable sheet side.

2. The diaper sensor according to claim 1, wherein
both the first water-absorbing sheet and the second water-absorbing sheet are included in the diaper sensor.

3. The diaper sensor according to claim 1, wherein
the wetness detection sensor device includes
a board having a base, a first conductive pattern formed on the base, and a mount component electrically connected to the first conductive pattern, and
a second conductive pattern forming the circuit together with the first conductive pattern in a complementary manner,
a portion of the base corresponding to the second conductive pattern is a missing portion where the base is missing, and
the second conductive pattern is soluble in liquid, or is weakened when getting wet with liquid.

4. The diaper sensor according to claim 3, wherein
the second conductive pattern includes multiple second conductive patterns, and
the missing portion of the base includes multiple missing portions each corresponding to the multiple second conductive patterns.

5. The diaper sensor according to claim 1, wherein
the diaper sensor is formed in a sheet shape.

6. A paper diaper comprising:
a paper diaper body;
a first diaper sensor provided at a portion of the paper diaper body corresponding to an external urethral opening; and
a second diaper sensor provided at a portion of the paper diaper body different from that for the first diaper sensor,
wherein each of the first diaper sensor and the second diaper sensor is the diaper sensor according to claim 1.

7. The paper diaper according to claim 6, wherein
the second diaper sensor is arranged at a peripheral edge portion of the paper diaper body.

8. A paper diaper comprising:
a paper diaper body; and
a diaper sensor provided at a portion of the paper diaper body different from a portion corresponding to an external urethral opening, the diaper sensor being the diaper sensor according to claim 1.

9. A paper diaper kit comprising:
a paper diaper; and
an incontinence pad,
the paper diaper comprising:
a paper diaper body; and
a diaper sensor provided at a portion of the paper diaper body different from a portion corresponding to an external urethral opening, the diaper sensor being the diaper sensor according to claim 1,
the incontinence pad has an incontinence pad body and a pad-side sensor device provided on the incontinence pad body, and
the pad-side sensor device is the diaper sensor.
